(19) United States
(12) Patent Application Publication
(10) Pub. No.: US 2011/0104278 A1
(43) Pub. Date: May 5, 2011

(54) Bone tissue substitute, a solidifiable bone precursor and method of making same

(76) Inventor: Sidney Omelon, Willowdale (CA)

(21) Appl. No.: 12/588,854

(22) Filed: Oct. 30, 2009

(51) Int. Cl. A61K 9/14; A61K 38/43; A61K 33/42; A61P 19/08

(52) U.S. Cl. 424/484; 424/94.1; 424/602; 424/604; 424/605

(57) ABSTRACT

The present concerns a solidifiable bone precursor composition which can be used to produce a mineralized substitute bone tissue material and the method of making the substitute bone tissue material. The substitute bone tissue material includes a collagen matrix comprising pores, and an apatitic mineral within the pores of general formula:

Aₛ(TOₓ)ₜ(QOₓ)ᵤZ_{y}

wherein A is a cation selected from Na, K, Mg, Ca, Fe³⁺, Al³⁺ or combinations thereof, T and Q are an anion oxide selected from P or C, Z is a H, OH or a halogen group, and s is 1 to 10, x is 2 to 4, t is 1 to 3, is 0 to 3 and y is 0 to 3. The solidifiable bone precursor composition includes: an aqueous dispersion comprising: at least one cation; and a polyphosphate combined to produce a cation-polyphosphate granule; and a collagen.

### TECHNICAL FIELD

The field of the present invention concerns a solidified bone precursor, which can be used to produce a mineralized bone tissue substitute.

### BACKGROUND OF THE INVENTION

Bone Mineral. Within the vertebrate skeleton, the mineral in bone apatite has a very small particle size (10's of Angstroms) is poorly crystalline and highly substituted and is interspersed within collagen (mostly type I collagen). Biological apatite is also carbonated, unlike geological apatites. “Interspersed”, is defined herein to mean that the apatite is in discrete particles segregated from each other by a hydrated organic matrix composed of type I collagen and other similar proteins. Apatite mineral can be considered as the ceramic component of the bone (brittle, good for compressive strength). Type I collagen can be considered the organic component (elastic, good for tensile strength), electrostatic forces, and a series of proteins that respond to electrostatic forces, can be considered the link between the mineral and organic phases of bone.

Bone. The vertebrate skeleton must satisfy a wide range of demands, including structural integrity, metabolic activity, growth, and continual repair of wear and damage caused by locomotion and/or trauma. These demands require that vertebrates continually resorb and rebuild their mineralized skeleton. “Remodelling” is the term used to describe this controlled destruction (resorption) and rebuilding. Newly formed bone (called osteoid) is a largely unmineralized collagenous matrix.

Mineralization of new, unmineralized skeletal tissue generally falls into two classes: intramembranous and endochondral ossification. Intramembranous ossification refers to the mineralization of newly formed osteoid. Osteoid is also what is formed to fill in the voids when bones break.

Because mineralized tissue is subject to damage and is metabolically active, mineralized bone is continually remodelled by the action of the basic multicellular unit (BMU) (Frost, H. M. Calcif. Tissue Res., 1969, 3, 211). The BMU is composed of two cell types: bone resorbing osteoclasts and bone building osteoblasts. In the accepted model of bone resorption, the osteoclasts form a sealed resorption zone at the bone surface. The acidic environment of this sealed-off zone generates a “resorption pit” as it dissolves the bone mineral, subsequently releasing enzymes into the pit to digest the exposed collagen. Once the sealed, ruffled border of the resorption zone is broken, the osteoclasts can migrate to form a new resorption zone elsewhere. Curiously, the dissolved apatite mineral does not spontaneously re-precipitate within the resorption pit, even when the newly reopened zone returns to a neutral pH. The void left by the excavating osteoclasts is filled with new bone formed by the osteoblasts. These bone-building cells lay down osteoid that in humans mineralizes 15 to 20 days later, a poorly understood period known as the mineralization lag time (Eriksen, E. F.; Axelrod, D. W.; Melson, F.; Bone Histomorphometry, 1994 [New York: Raven Press].

Bone Healing. Bone tissue that has been fractured can heal if the size of the defect is less that the “critical size”, and the fracture site is immobilized. Much work has gone into developing biomaterials that will “fill” a “critical-sized defect, as injuries involving a large loss of bone (such as complicated fractures or osteotomies) are often only best assisted by the implantation of host bone tissue harvested from non-critical bone masses such as the iliac crest.

Many materials proposed to fill these critical-sized defects are composed of materials that can be resorbed and remodelled by the body, as human bone tissue has remarkable material properties that cannot be replicated by engineered materials. The inspiration of many of these materials is the mineral found in bone: apatite. Often, many people in the field use hydroxyapatite (Ca₅(OH)(PO₄)₃), a member of the apatite mineral family.

As such, bone mineral analogues includes hydroxyapatite (Ca₅(OH)(PO4)₃) and octacalcium phosphate (Ca₈(HPO₄)(PO₄)·5H₂O) that have been found to be unsatisfactory. Therefore, until very recently the means by which bone is solidified through mineralization was unknown. Of interest to the biomaterial community would be an apatite mineral similar to that found in bone tissue. Of further interest would be a composite biomaterial that mimics bone, that has an organic and a mineral component.

### SUMMARY OF THE INVENTION

The present invention describes the formation of a bone tissue substitute material comprising a cation phosphate mineral, ideally apatite, from a calcium polyphosphate precursor, which is embedded within an organic matrix, such as type I collagen from a solidifiable bone precursor. The resultant bone tissue substitute composite material mimics bone tissue precursor material (osteoid). The bone substitute material of the present invention may be produces in a variety of forms from soft (and unformed) to hard and completely mineralized; and all variations between.

In accordance with one aspect of the present invention, there is provided a bone tissue substitute, comprising: a collagen matrix comprising pores, and an apatitic mineral within the pores having the general formula: Aₛ(TOₓ)ₜ(QOₓ)ᵤZ_{y}, wherein A is a cation selected from the group consisting of Na, K, Mg, Ca, Sr, Fe³⁺, Al³⁺ and combinations thereof, T is an anion oxide selected from P or C, Q is an anion oxide selected from P or C, Z is a hydroxyl or halogen group, and s is 1 to 10, x is 2 to 4, t is 1 to 3, u is 0 to 3, and y is 0 to 3.

In accordance with another aspect of the present invention, there is provided a bone tissue precursor material and a method to produce a precursor to mineralized bone formation. In a preferred embodiment the material precursor is an organic matrix interspersed with the major apatite generators, these generators comprise mainly calcium and polyphosphate.

In accordance yet another aspect of the present invention, there is provided a bone tissue substitute material, the material comprising: an aqueous dispersion comprising at least one cation and a polyphosphate combined to produce a cation-polyphosphate granule and a collagen, wherein the complex is dispersed and forms an apatitic mineral within the organic matrix solidifying the material mineralized bone substitute.

In accordance with still another aspect of the present invention, there is provided a method of making a substitute bone tissue material comprising steps of: preparing an aqueous dispersion comprising: a cation; and a polyphosphate to produce a cation polyphosphate granule; and contacting the granules into collagen, to disperse the granules within the collagen.

In accordance with yet still another aspect of the present invention, there is provided the method of making a bone tissue precursor composition comprising the steps of:—preparing an aqueous dispersion comprising a cation, a polyphosphate and an enzyme or protein to produce cation polyphosphate granules that are at least within and on the surface of the cation polyphosphate granules;—providing an organic matrix which may or may not contain enzymes, proteins, or other molecules that bond electrostatically or by other means to the cation polyphosphate granules that may or may not contain and/or exhibit enzymes, proteins, or other molecules on the surface of the cation polyphosphate granules; and—dispersing the enzyme-cation polyphosphate granules that may or may not contain enzymes, proteins, or other molecules into an organic matrix that may or may not contain enzymes, proteins, or other molecules that bond electrostatically or by other means to the cation polyphosphate granules.

In accordance with a further aspect of the present invention, there is provided the use of the substitute bone tissue material.

In accordance with yet a further aspect of the present invention, there is provided a bone tissue precursor material further comprising a polyphosphate hydrolytic degradation agent, wherein the agent transforms the cation-polyphosphate complex into an apatitic mineral. Preferably, the polyphosphate hydrolytic degradation agent is a phosphatase enzyme, such as, an alkaline phosphatase. However, the polyphosphate hydrolytic degradation agent may be an inorganic catalyst, such as acid or a base, such as respectively hydrochloric acid and sodium hydroxide.

In accordance with still further aspect of the present invention, the bone tissue precursor material comprises cation-polyphosphate granules and a buffer agent to maintain a pH conducive to the precipitation of apatitic minerals from the orthophosphates produced by the hydrolytic degradation of the polyphosphates. This buffer may comprise carbonate species, such as bicarbonate or carbonate ions.

In accordance with yet still a further aspect of the present invention, the bone tissue precursor material comprises cation-polyphosphate granules that may be encapsulated by an organic compound. This organic compound may comprise lipid bilayers, and may include one or more active enzymes such as at least one of a phosphatases and a carbonic anhydrase.

In still another aspect of the invention there is provided a method of making a bone tissue precursor material comprising steps of: providing cation polyphosphate granules, and dispersing the granules into a collagen.

In yet still another aspect of the invention, the cation polyphosphate granules are partially or whole transformed into apatitic minerals while dispersed into an organic matrix.

### BRIEF DESCRIPTION OF THE DRAWINGS

FIG. 1 is a powder X-ray diffractogram of a precursor bone material containing calcium, polyphosphate, and collagen according to one embodiment of the present invention;

FIG. 2 illustrates a schematic reaction of a calcium-polyphosphates, catalyzed by alkaline phosphatase (ALP), with water to produce calcium and orthophosphate (Pi) ions according to one embodiment of the present invention;

FIG. 3 is an X-ray diffraction pattern of solids precipitated from a precursor material according to one embodiment of the present invention identifying, halite (NaCl), Nahcolite (NaHCO₃), hydroxylapatite (Ca₅(OH)(PO₄)₃) and/or carbonated hydroxylapatite (Ca₁₀(PO₄)₃(CO₃)₃(OH)₂); and

FIG. 4 is an X-ray diffraction pattern of solids precipitated from a precursor material to another embodiment of the present invention identifying halite (NaCl), Nahcolite (NaHCO₃), hydroxylapatite (Ca₅(OH)(PO₄)₃) and/or tuite (Ca₃(PO₄)₂).

### DETAILED DESCRIPTION

The cation-polyphosphate-collagen composite material, or bone tissue precursor material of the present invention is meant to be transformed into a mineralized bone substitute. Previously known bone substitutes involve the use of calcium phosphate minerals, including hydroxyapatite (Ca₅(OH)(PO₄)₆).

The bone tissue precursor material produced by the present method is defined as an “un-mineralized” matrix composed of organic long-chain molecules and water, includes the organic matrix, is preferably a collagen, more preferably, type I, collagen and having embedded within the matrix, a cation-polyphosphate complex material. The cations of the polyphosphate are preferably magnesium and/or calcium.

The cation-polyphosphate granules within the bone tissue precursor material are generally amorphous, and contain the ions required to transform into a mineralized bone substitute which is generally an apatitic mineral, where the general apatite structure is defined as Aₛ(TOₓ)ₜ(QO)ᵤZ_{y} wherein A is a cation selected from the group consisting of Na, K, Mg, Ca, Sr, Fe³⁺, Al³⁺ and combinations thereof, T is an anion oxide selected from P or C, Q is an anion oxide selected from P or C, Z is a hydroxyl or halogen group, and s is 1 to 10, x is 2 to 4, t is 1 to 3, u is 0 to 3 and y is 0 to 3. A common example is hydroxyapatite (Ca₅(OH)(PO₄)₃).

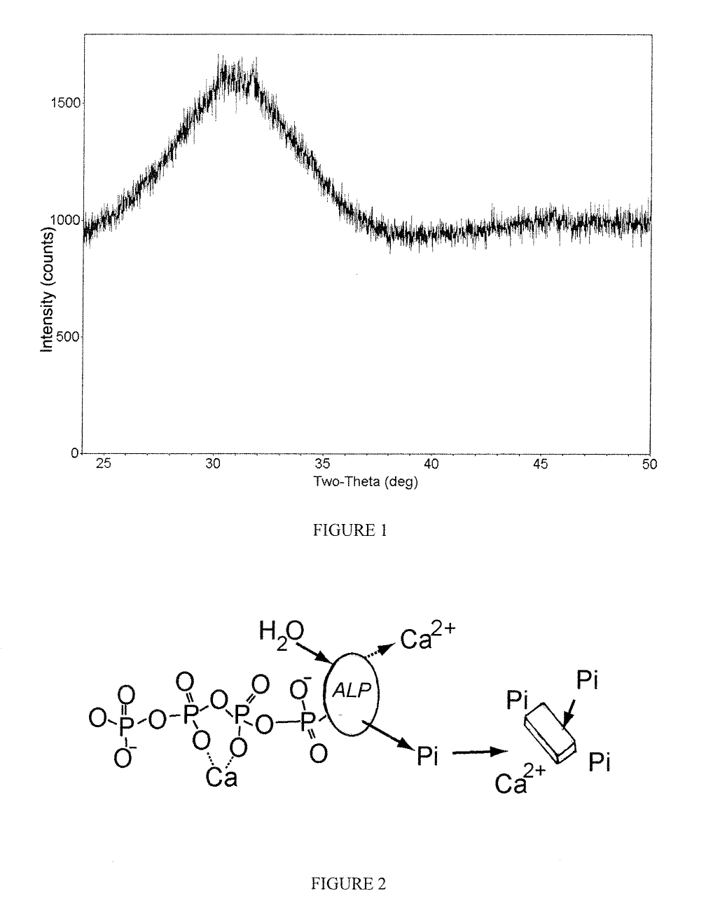

The amorphous nature of the cation-polyphosphate-organic material is detected by powder x-ray diffraction, as exemplified by FIG. 1. In this case, the starting materials were sodium hexametaphosphate mixed with calcium chloride, which formed a calcium-polyphosphate coascervate which was removed from solution and then mixed with collagen. This dried material was ground and analyzed by powder x-ray diffraction. The powder x-ray diffraction pattern shows one wide curve, with no discernable peaks, which exemplifies a solid material with no crystalline structure.

Acidification of the amorphous precursor material with 7% acetic acid accelerated the hydrolytic degradation of polyphosphates into orthophosphates (FIG. 2).

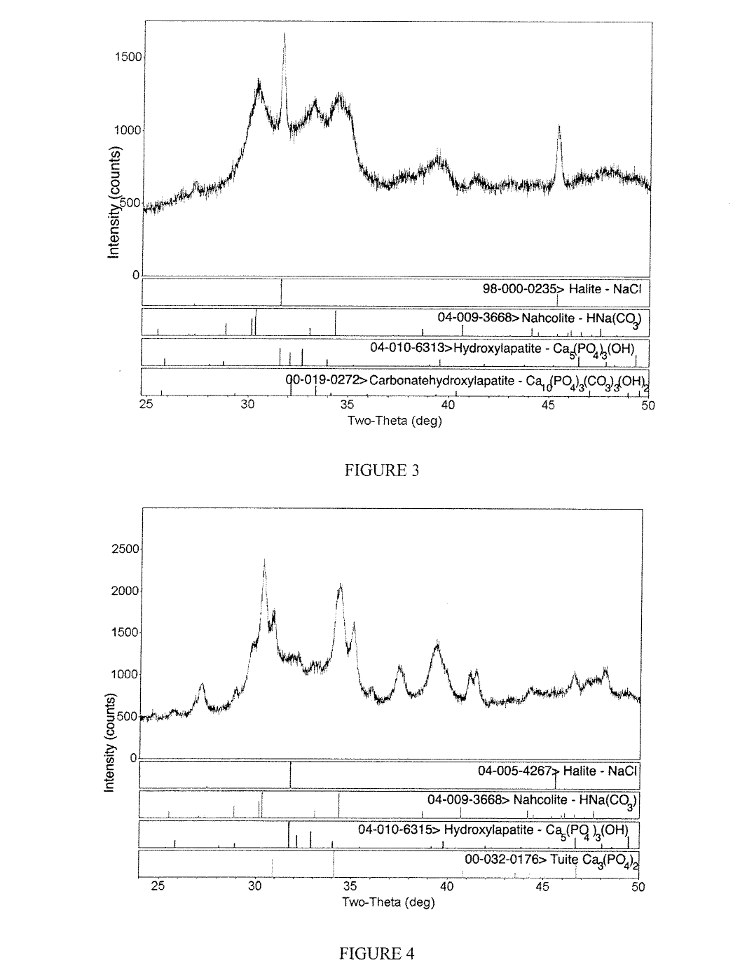

The calcium-and phosphate-containing solution can precipitate one and/or many calcium phosphate phases if the solubility product of that calcium phosphate crystal phase is exceeded. The apatitic crystal phases are favored to precipitate at neutral to basic pH's. Mixing the calcium-and-phosphate-containing solution with a sodium-bicarbonate solution favors the formation of basic calcium-phosphate phases such as hydroxyapatite, and possibly carbonated hydroxyapatite. FIGS. 3 and 4 show examples of powder x-ray diffraction spectra of the solid products dried from the solution composed of acidified calcium-polyphosphate and collagen material mixed with a sodium bicarbonate solution. The powder x-ray diffraction patterns in FIGS. 3 and 4 show diffraction peaks that were identified as halite (NaCl), nahcolite (NaHCO₃), hydroxylapatite (Ca₅(OH)(PO₄)₃) and/or carbonated hydroxylapatite Ca₅(PO₄)₂(CO₃)(OH)₂; Mg₅(PO₄)(CO₃)(OH₂); Ca₅(PO₄)₃(CO₃), and/or tuite (Ca₃(PO₄)₂).

The transformation of this bone tissue precursor material into a mineralized bone substitute material that can be implanted into a patient in need. However, the implantation of the bone tissue precursor material may be after complete or partial transformation of the precursor into the mineralized bone substitute. However, the bone tissue precursor material may also be implanted before transformation, with transformation from the cation-polyphosphate to the apatitic mineral occurring in vivo.

The mineralized bone substitute in large part been mineralized to an apatitic mineral, these apatitic minerals are understood to include calcium and/or magnesium, fluoride phosphate, Ca₅F(PO₄)₃, where chlorine, hydroxyl, or carbonate sometimes replacing the fluoride, where in a simplified formulation hydroxyl apatite is represented by Ca₅(PO₄)₃OH, and where hydroxyl apatite is a preferred embodiment. These apatitic mineral are understood to provide the component that exhibits compressive strength of vertebrate bone—a composite material of mineral and organic phases.

This transformation of the bone tissue precursor into a mineralized bone substitute may be undertaken in vivo by the action of in situ phosphatase enzymes, or the thermodynamically favoured hydrolytic degradation of polyphosphates into orthophosphates with water.

The method of making the bone tissue precursor material includes the:

production of calcium and/or magnesium or other polyphosphate granules or spheres in a preferred size range from 100 to 10,000 nm. This production process may be executed through the controlled introduction of cation and polyphosphate ions into an organic solution with organic components under specific conditions as to result in the formation of cation-polyphosphate-containing spheres of desired size, encapsulated with a specific organic material or by the precipitation of polyphosphate ions within an organic matrix such as an acidic gelatin (Griffith ibid., p. 87). In a preferred embodiment the cation of the polyphosphate is calcium;

the polyphosphate spheres may optionally be encapsulated with an organic layer; the organic layer may or may not be a lipid and may or may not include enzymes such as alkaline phosphatase and/or carbonic anhydrase;

dispersing and incorporating the polyphosphate granules or spheres within a collagen matrix, typically a collagen material, and preferably type I collagen; and

the bone tissue precursor material composition that is solidifiable may or may not include native bone marrow and/or native bone particles further dispersed within the bone tissue precursor material or matrix. “Solidifiable” is understood to mean that the precursor becomes hard and mineralizes.

The organic matrix is a hydrogel-like material comprised of organic, usually long-chain molecules that form a network that results in a material with a viscosity greater than water. These long-chain molecules comprise but are not limited to a mixture fibrous protein, deriving from constituents such as bone, cartilage, tendon and/or other connective tissue.

The composition may also comprise:
a polyphosphate that is a linear condensed phosphate polymeric anion a length from two and 100,000 phosphate units;
an aqueous dispersion comprises water and salt, where the salt is selected from the group consisting of sodium chloride, magnesium chloride, potassium chloride, sodium bicarbonate, potassium bicarbonate and combination;
a polyphosphate hydrolytic degradation agent. wherein the agent accelerates the transformation of the cation-polyphosphate complex into the apatitic mineral; where in a preferred embodiment the polyphosphate hydrolytic degradation agent is a phosphatase enzyme; more preferably the phosphatase enzyme is an alkaline phosphatase.
a buffer, wherein the buffer neutralizes pH changes induced by the polyphosphate hydrolytic degradation process, and/or the apatite precipitation process into the apatitic mineral, wherein in a preferred embodiment the buffer is selected from the group consisting of carbonate, bicarbonate and mixtures thereof; more preferably the buffer is produced by action of a carbonic anhydrase enzyme; where more preferably the polyphosphate hydrolytic degradation agent is an acid or a base; where in another embodiment the polyphosphate hydrolytic degradation agent is an inorganic catalyst of the hydrolytic degradation of polyphosphate ions into orthophosphate ions.

The bone tissue precursor material may be used by:
using a cation-polyphosphate-organic matrix composite material as a filler of voids within bone defects; in a preferred embodiment the cation-polyphosphate granule has a diameter between 10 and 100,000 nm, preferably between or 100 and 10,000 nm, and most preferably 100-100 nm. In another embodiment the cation-polyphosphate granule may be encapsulated by the collagen,
partially or wholly transforming the cation-polyphosphate-organic matrix composite material into an apatitic-organic matrix composite material for use as a filler of voids within bone defects,
incorporating the cation-polyphosphate- and/or apatitic-organic matrix with other mineralized bone substitutes or bone pieces, such that the bone tissue precursor material or matrix acts as a mortar to hold together and/or assist in the filling of voids within bone defects;
temporarily placing the bone tissue precursor material on or within a metal or phosphate glass support or mold structure for an initial placement on which the bone tissue precursor material may then be completely or partially converted to the mineralized bone substitute; and
optionally the formation of the mineralized bone substitute can be accelerated by using the polyphosphate hydrolytic degradation agent, to accelerate the hydrolytic degradation of the polyphosphates, producing orthophosphate ions and favouring the apatite precipitation reaction within the calcium-polyphosphate/collagen matrix through the application of heat, water, inorganic catalysts, or the application of phosphatase enzymes, within a slightly acidic, generally neutral or slightly basic (ranging from pH 6 to 11).

In a preferred embodiment, the cation polyphosphate granules or spheres dispersed into the organic matrix are in a size range of (100-1000 nm). The organic matrix is preferably collagen type I.

The collagen may also include comprises lipid bilayers, where in a preferred embodiment the lipid bilayer includes one or more active enzymes, that are activated after implanted within a patient that catalyze or otherwise participate in the hydrolytic degradation of polyphosphate to orthophosphate and/or the maintenance of an environment suitable for the formation of apatitic minerals from cation-polyphosphate precursors, and more preferably the one or more active enzymes, include enzymes.

As the bone tissue precursor material is a deformable hydrogel, it has the advantage of being easily injected in spaces or molds and thus can be formed into any desired shape, or can serve as a bone void filler material.

The bone tissue precursor material composition may alternatively be supported by resorbable structures and/or inert such as a phosphate glass structure, organic polymeric structure, and/or metal structure.

Importantly, the composition may be mixed in vivo with a sourced bone tissue precursor composition.

The bone precursor composition uses the thermodynamic driving force for water to hydrolytically degrade polyphosphate ions into orthophosphate ions in an environment where the orthophosphate ions will react with cations to form an apatitic mineral within an organic phase;
wherein in a preferred embodiment the environment in which the polyphosphate hydrolytic degradation causes the incomplete or complete formation of apatite from cation-polyphosphate precursors in an in vitro environment;
wherein in another embodiment the environment in which the polyphosphate hydrolytic degradation causing the incomplete or complete formation of apatitic within an in vitro environment involves the secondary addition of an inorganic and/or organic agent that catalyzes the transformation of the polyphosphate component of the bone precursor material partially or totally into an apatitic material, and this catalytic material is distributed within the material by mechanical agitation and/or diffusion.

1. A bone tissue substitute, comprising:
a collagen matrix comprising pores, and an apatitic mineral within the pores having the general formula:
Aₛ(TOₓ)ₜ(Q0ₓ)ᵤZ_{y}
wherein A is a cation selected from the group consisting of Na, K, Mg, Ca, Sr, Fe³⁺, Al³⁺ and combinations thereof,
T is an anion oxide selected from P or C,
Q is an anion oxide selected from P or C,
Z is a hydroxyl or halogen group, and
s is 1 to 10,
x is 2 to 4,
t is 1 to 3,
u is 0 to 3, and
y is 0 to 3.

2. The substitute of claim 1, wherein A is selected from the group consisting of Na, K, Mg, Ca, and combinations thereof.

3. The substitute of claim 2, wherein the T is P and x is 4.

4. The substitute of claim 3, wherein A is Ca.

5. The substitute of claim 1, comprising (Ca₁₀(PO₄)₃(CO₃)₃(OH)₂).

6. The substitute of claim 1, comprising a salt.

7. A solidifiable bone precursor composition comprising:
an aqueous dispersion comprising:
at least one cation; and
a polyphosphate combined to produce a cation-polyphosphate granule; and
a collagen,
wherein the complex is dispersed and forms an apatitic mineral within the organic matrix solidifying the composition.

8. The composition of claim 7, further comprising the apatitic mineral, having the general formula:
Aₛ(TOₓ(QOₓ)ᵤZ_{y}
wherein A is a cation selected from the group consisting of Na, K, Mg, Ca, Sr, Fe³⁺, Al³⁺ and combinations thereof,
T is an anion oxide selected from P or C,
Q is an anion oxide selected from P or C,
Z is a hydroxyl or halogen group, and
s is 1 to 10,
x is 2 to 4,
t is 1 to 3,
u is 0 to 3, and
y is 0 to 3.

9. The composition of claim 7, wherein the apatitic mineral is formed from an orthophosphates sourced from the hydrolytic degradation of polyphosphates, that comprises combination of cations, oxide anions and hydroxide.

10. The composition of claim 7, where the cation is at least one of a monovalent cation, divalent cation and a trivalent cation.

11. The composition of claim 10, wherein the monovalent cation is selected from the group consisting of sodium and potassium, the divalent cation is selected from the group consisting of magnesium, and calcium, and the trivalent cation is selected from the group consisting of ferric iron and aluminum.

12. The composition of claim 7, wherein the polyphosphate is a linear condensed phosphate polymeric anion a length from two and 100,000 phosphate units.

13. The composition of claim 7, wherein the aqueous dispersion comprises water and salt.

14. A method of making a substitute bone tissue material comprising steps of:
preparing an aqueous dispersion comprising:
a cation; and
a polyphosphate to produce a cation polyphosphate granule; and
contacting the granules into collagen, to disperse the granules within the collagen.

15. The method of making a bone tissue precursor composition comprising the steps of:
preparing an aqueous dispersion comprising a cation, a polyphosphate and an enzyme or protein to produce cation polyphosphate granules that are at least within and on the surface of the cation polyphosphate granules;
providing an organic matrix which may or may not contain enzymes, proteins, or other molecules that bond electrostatically or by other means to the cation polyphosphate granules that may or may not contain and/or exhibit enzymes, proteins, or other molecules on the surface of the cation polyphosphate granules; and
dispersing the enzyme-cation polyphosphate granules that may or may not contain enzymes, proteins, or other molecules into an organic matrix that may or may not contain enzymes, proteins, or other molecules that bond electrostatically or by other means to the cation polyphosphate granules.
